(19) United States
(12) Patent Application Publication
(10) Pub. No.: US 2012/0130310 A1
(43) Pub. Date: May 24, 2012

(54) Joint pressure measurement with sensor in the drainage line

(75) Inventors: Martin Reuther, Berlin (DE); Matthias Stiller, Berlin (DE); Thomas Merzhauser, Berlin (DE)

(73) Assignee: W.O.M. WORLD OF MEDICINE AG, Berlin (DE)

(21) Appl. No.: 13/253,246

(22) Filed: Oct. 5, 2011

(30) Foreign Application Priority Data
Oct. 5, 2010 (DE) 10 2010 047 349.9

(51) Int. Cl. A61M 3/02

(52) U.S. Cl. 604/151

(57) ABSTRACT

The invention relates to a device for rinsing a body cavity, wherein a pressure sensor for determining the actual pressure in the body cavity is provided, said pressure sensor being arranged in the drainage line (8).

### FIELD OF THE INVENTION

The invention relates to a device for rinsing a body cavity with a fluid, comprising a storage container for the fluid, comprising a controllable rinsing pump connected to the storage container, comprising a feed line connected to the pressure side of the rinsing pump (3) and comprising a first medical instrument connected to the feed line and having a rinsing channel, said first medical instrument being capable of being introduced into the body cavity (1), comprising a drainage cannula (7) being capable of being introduced into the into the body cavity (1) and having a drainage line (8), in or at the drainage line (8) a pressure sensor (10) for determining the actual pressure being provided, the drainage line (8) being connected to a suction pump (9), a method for operating such a device and the use of such a device according to the invention.

Such devices are for instance used for endoscopic examinations, distension or resection of tissues, in particular under endoscopic control. A body cavity (1) may be a joint cavity, e.g. in a knee joint or shoulder joint, a cavity between muscles or organs, or also an organ itself forming or comprising a cavity. A fluid is in particular a liquid. It may be a homogeneous liquid phase, for instance a common salt solution, or also a dispersion or emulsion. A drainage cannula (7) in the meaning of the invention may fulfill, beside the drainage function, other functions, too, such as for instance an illumination of the body cavity (1) by a light source integrated in the drainage cannula (7). Different volume flows in different operating positions are distinguished in the invention by the terms “high” and “low”, the absolute values being irrelevant. Rather, these terms only designate, how the volume flows are relative to each other in the respective operating positions of the second medical instruments.

### PRIOR ART

Devices of the species mentioned above are for instance known in the art by the documents U.S. Pat. No. 4,902,277, U.S. Pat. No. 5,000,733, EP 0306445, EP 1382291 or U.S. Pat. No. 6,024,720. The difficulty in the prior art devices is the measurement of the pressure in the body cavity (1). This value is critical, since a too high pressure can lead to irreversible tissue damages. In the prior art devices, for instance, a pressure sensor is introduced into the body cavity (1), in addition to the feed line, output line and medical instrument, which leads to an increased risk of infection, but may also lead to obstacles to work.

In alternative embodiments, a pressure sensor is provided on the pressure side of the feed pump (see e.g. EP 1382291). It has been found, however, that the pressure measurement is difficult, since the pressure measured in the feed line depends on the flow resistance of the first medical instrument. In the practice, therefore, corrections of the measured pressure have to be made, the correction factors depending on the selection of the medical instrument. This leads to problems in the application of such devices.

### TECHNICAL OBJECT OF THE INVENTION

It is therefore the technical object of the invention to specify a device for rinsing a body cavity (1) with a fluid, which allows a pressure measurement in the body cavity (1), without the need of additional feed lines, and without the pressure measurement being dependent on the used medical instruments.

### BASICS OF THE INVENTION AND PREFERRED EMBODIMENTS

For achieving this technical object, the present invention teaches to provide a pressure sensor in the drainage line (8). The drainage line (8) further comprises a pinch valve (11) that can block the drainage line (8) from the suction side of the suction pump (9). For determining the pressure in the body cavity, the pinch valve is closed, and the pressure sensor (10) immediately shows the pressure in the body cavity (1).

The rinsing pump (3) may comprise a drive unit with a motor having a rotating drive shaft and a pump unit, being (mechanically) connected, in particular by bonding, form-fitting or force-fitting, or magnetically, in particular by force-fitting, with the drive shaft, and being rotatably driven, the capacity being controllable by controlling the speed of the motor. In particular, insofar, a peristaltic roller pump can be used. Alternatively, the rinsing pump (3) may comprise an elastic storage container with a controllable pressure sleeve partially or fully covering the storage container, the capacity being controllable by controlling the pressure sleeve. Finally, also alternatively, the rinsing pump (3) may comprise a height-controllable storage container, the capacity being controllable by controlling the height of the storage container.

As a suction pump, too, a peristaltic roller pump can be used.

The medical instrument may be selected from the group consisting of rinsing probe, trocar with optical system, optical system with rinsing cannula, rinsing cannula, shaver. The pinch valve (11) according to the invention is a hose clamping device, the hose wall of which is made of an elastic material, and the valve comprises a support surface, on which the hose wall of the hose rests, and comprises a pressure piece, by means of which a pressure can be exerted on the hose wall in the direction of the support surface. It is preferred that the pressure piece can be driven linearly and is connected by a spindle gear box with an electromotor drive, preferably a stepper motor.

The drainage line or the suction pump, resp., finally ends in a collection vessel (12).

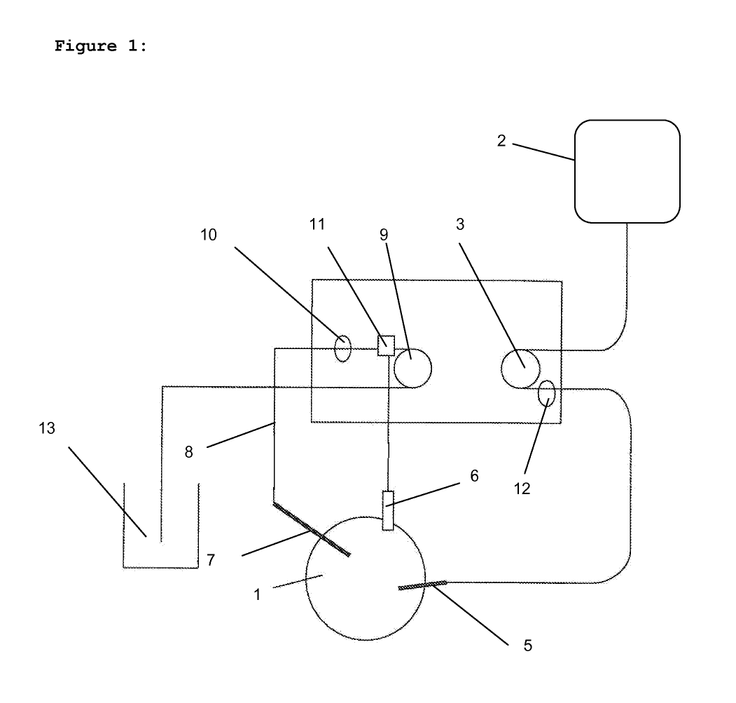

In the following, the invention is explained in more detail with reference to FIG. 1:

In FIG. 1 can be seen a device for rinsing a body cavity (1) with a fluid. This device is equipped with a storage container (2) for the fluid and with a rinsing pump (3) connected to the storage container. On the pressure side of the rinsing pump (3), a feed line (4) is connected. The feed line is provided with a first medical instrument with a rinsing channel (5), which medical instrument can be introduced into the body cavity (1). On the pressure side of the rinsing pump (3) is provided an optional additional pressure sensor (12) for determining an actual pressure.

Further, a drainage line (8) is shown, which can be introduced into the body cavity. The drainage line (8) is provided with the sensor according to the invention. In the flow direction downstream of the sensor, a pinch valve (11) is connected, which decouples the drainage line (8) from the suction pump. At the exit side of the suction pump (9), another collection vessel (13) is arranged.

In an improvement of the device according to the invention, a second medical instrument (6) is provided, for instance a shaver. During the operation of the second medical instrument (6), the pinch valve (11) is automatically closed, so that the volume flow of the fluid through the drainage line (8) is reduced. At this time, the pressure sensor (10) measures the pressure in the body cavity (1). When the shaver is turned off, the pinch valve (11) will automatically open, so that the body cavity (1) is rinsed. If desired, the pressure in the body cavity (1) can be measured during the rinsing process by the optional second pressure sensor (12).

### EXAMPLE

The accuracy of the measurement by the device according to the invention was determined by using a dummy. The dummy has in the body cavity a pressure sensor displaying the actual pressure in the body cavity. The pressure measurement of the device according to the invention in the drainage line (8) excellently matches the actual pressure, as soon as pinch valve is closed, and the flow through the drainage line (8) returns to zero. With opened pinch valve and during pump operation, there are of course different pulse phases depending on the peristaltics of the pump.

The use of the device according to the invention results in that the pressure limits can be set to substantially higher values. This permits a higher flow rate and thus better rinsing conditions in the body cavity with an overall improved visibility.

Optionally, the device according to the invention may comprise a second sensor (12) on the pressure side of the rinsing pump. This second sensor is in particular used, when the pinch valve has to be opened, e.g. during the rinsing phases of medical treatments.

1. A device for rinsing a body cavity with a fluid, comprising a controllable rinsing pump with a feed line connected to the pressure side of the rinsing pump and comprising a first medical instrument with a drainage cannula and comprising a drainage line with a pinch valve, comprising an optional second pump for extracting the fluid from the drainage line, wherein the drainage line upstream of the pinch valve, a pressure sensor for determining the pressure in the body cavity is provided.

2. The device according to claim 1, wherein the first medical instrument is selected from the group consisting of rinsing probe, trocar with optical system, optical system with rinsing channel, rinsing cannula, shaver.

3. The device according to claim 1, further comprising a second medical instrument.

4. The device according to claim 3, wherein the second medical instrument is a shaver.

5. The device according to claim 4, wherein the shaver controls the pinch valve such that when setting the shaver into operation, the pinch valve moves to the position “closed”, and when the shaver is at a standstill, the pinch valve moves to the position “open”.

6. The device according to claim 1, wherein a second pressure sensor is connected to the feed line on the pressure side of the rinsing pump.

7. The device according to claim 1, wherein the pressure measurement is made with the pressure sensor when the pinch valve is closed.

8. The device according to claim 6, wherein the pressure measurement is made with the pressure sensor, when the pinch valve is closed, and that the pressure measurement is made with the second pressure sensor, when the pinch valve is open.
